(19) United States
(12) Patent Application Publication
(10) Pub. No.: US 2017/0232157 A1
(43) Pub. Date: Aug. 17, 2017

(54) TISSUE INTEGRATING MATERIALS FOR WOUND REPAIR

(71) Applicant: Arizona Board of Regents on behalf of Arizona State University, Scottsdale, AZ (US)

(72) Inventors: Kaushal Rege, Chandler, AZ (US); Taraka Sai Pavan Grandhi, Tempe, AZ (US); Jerry Crum, Phoenix, AZ (US); Russell Urie, Tempe, AZ (US)

(21) Appl. No.: 15/430,234

(22) Filed: Feb. 10, 2017

Related U.S. Application Data
(60) Provisional application No. 62/294,226, filed on Feb. 11, 2016.

(51) Int. Cl. A61L 31/12; A61L 31/16; A61L 31/14; A61B 17/06; A61B 17/064

(52) U.S. Cl. CPC A61L 31/128; A61B 17/06166; A61B 17/064; A61L 31/14; A61L 31/148; A61L 31/16; A61B 2017/00876

(57) ABSTRACT

A tissue closure device can include a structural material and a stimulus responsive material on or in the structural material. The structural material can be biodegradable and/or bioabsorbable (e.g., biocompatible natural and/or semi-natural and/or synthetic polymer). The stimulus responsive material can be a particle, such as a nanoparticle. The structural material is shaped as a suture, staple, screw, patch, adhesive, sealant, or the like. A biologically active agent can be included. A method of promoting wound healing can include: approximating tissue portions; and stimulating the stimulus responsive material with a stimulus to cause the tissue portions of the wound to adhere to each other. The stimulus is selected from optical, electrical, thermal, chemical, mechanical, magnetic, acoustic, pressure, shear, biological, or enzymatic sources.

### CROSS-REFERENCE

This patent application claims priority to U.S. Provisional Application No. 62/294,226 filed Feb. 11, 2016, which provisional is incorporated herein by specific reference in its entirety.

### GOVERNMENT RIGHTS

This invention was made with government support under R01 EB020690 awarded by the National Institutes of Health. The government has certain rights in the invention.

### BACKGROUND

Surgical repair of wounds or openings in body tissues using sutures or other closure means (e.g., staples etc.) are longstanding treatments that have changed very little in recent years. However, sutures and other closure means may not be suitable for use in friable tissue or other tissues or wound types that are difficult to close. Also, suture alternatives, such as staples, nitinol clamps, and surgical adhesives, have not overcome some of the difficulties experienced with sutures, and may have even exacerbated some of the drawbacks of conventional sutures.

Laser tissue welding is a platform technology that has been researched as an alternative to sutures. In laser tissue welding, an exposed chromophore converts laser light to heat to rapidly seal tissue wounds or incisions. With the use of exogenous chromophores in laser tissue welding materials, laser irradiation can be employed at wavelengths of 650-1350 nm; however, tissue absorbance at this wavelength is lowest for light in the near infrared range (700-1000 nm wavelength).

General current state of the art in sutures/other closure methods can include the following types and associated issues. Triclosan-coated sutures still are sutures (e.g., traumatic and must puncture the tissue multiple times) and can have leakage or dehiscence, and the sutures do not integrate with the tissue. Staples require removal; can have leakage, trauma, and inflammation; and may result in greater scarring. Fibrin glue is brittle when cured, may cause problems with sequestering of bacteria, and is not suitable for internal applications. Sealants and adhesives require curing times, which can be long or require a UV light that may be harmful to cells, and typically are used over a sutured closure, and thereby are not standalone products. Albumin solder and other solders for laser tissue welding are liquid systems with inconsistent reproducibility, and they use organic dyes as a chromophore within a liquid, which results in rapid loss of chromophore stability due to photobleaching, and also results in leaching of the chromophore to surrounding tissue, which is not beneficial.

However, wound repair continues to be a surgical necessity, and research into improved wound repair is desirable. Therefore, it would be advantageous to have a system for improving surgical repair of wounds that can overcome the limitations of traditional closure means.

### SUMMARY

In one embodiment, a tissue closure device can be adapted to close an opening in a tissue. Such a device can include a structural material and a stimulus responsive material on or in the structural material. In one aspect, the structural material is biodegradable and/or bioabsorbable. In one aspect, the structural material includes a biocompatible natural and/or semi-natural and/or synthetic polymer. In one aspect, the structural material includes nylon, rayon, polyethylene, pluronic F127 (poloxamer 407), chitosan, collagen, laminin, fibronectin, polyacrylamide, aminoglycoside hydrogels, fibrin, poly-lactic acid, poly-glycolic acid, poly-lactic-co-glycolic acid, polyglyconate (Maxon), polydioxanone (PD S), silk, poly-glycolic-caprolactone, cotton, gelatin, polypropylene (prolene), titanium, metal, polysulfone, copolymers thereof, or others. In one aspect, the stimulus responsive material is coated, embedded, crosslinked, or otherwise associated with the structural material. In one aspect, the stimulus responsive material is in a particle form, such as when the stimulus responsive particle is a nanoparticle (e.g., nanosphere, nanorod, etc.). In one aspect, the structural material is shaped as a suture, staple, screw, patch, adhesive, sealant, or the like. In one aspect, the tissue closure device can include a biologically active agent in the structural material.

In one embodiment, a method of promoting wound healing can include: providing a tissue closure device of one of the embodiments; approximating tissue portions of a wound with the tissue closure device; and stimulating the stimulus responsive material with at least one stimulus so as to cause the tissue closure device to change a property so that the tissue portions of the wound adhere to each other and/or to the tissue closure device in response to the property change. In one aspect, the stimulus is selected from optical, electrical, thermal, chemical, mechanical, magnetic, acoustic, pressure, shear, biological, or enzymatic stimulus applied to the tissue closure device. In one aspect, the method includes stimulating the stimulus responsive material to generate heat that causes tissue components of the tissue portions to interdigitate. In one aspect, the method includes stimulating the stimulus responsive material to induce a chemical reaction that causes tissue components of the tissue portions to chemically or physically interact with each other. In one aspect, the method includes stimulating the stimulus responsive material to cause the tissue portions to weld and seal the wound. In one aspect, the method includes eluting a biologically active agent from the tissue closure device into the wound. In one aspect, the method includes causing tissue integration of the tissue portions.

In one embodiment, a method can be used for making the tissue closure device of one of the embodiments. Such a method can include: obtaining the structural material; obtaining the stimulus responsive material; combining the structural material and the stimulus responsive material; and obtaining the tissue closure device having the structural material and stimulus responsive material.

In one embodiment, a method can be used for making the tissue closure device of one of the embodiments. Such a method can include: obtaining the structural material having the shape of a tissue closure device; coating the structural material with the stimulus responsive material; and obtaining the tissue closure device having the stimulus responsive material coated on the structural material.

In one embodiment a stimulus responsive tissue glue composition can include a tissue adhesive that adheres to tissue and a stimulus responsive material in the tissue adhesive. In one aspect, the tissue adhesive is a cyanoacrylate.

In one embodiment, a method of promoting wound healing is provided. Such a method can include: providing a stimulus responsive tissue glue composition of one of the claims; approximating tissue portions of a wound with the stimulus responsive tissue glue composition; and stimulating the stimulus responsive material with at least one stimulus so as to cause the tissue portions of the wound to adhere to each other and/or to the stimulus responsive tissue glue composition.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through the use of the accompanying drawings, in which:

FIG. 1A shows an embodiment of a closure device in the form of a suture.

FIG. 1B shows an embodiment of a closure device in the form of a staple.

FIG. 2 shows an embodiment of a method of using a closure device to approximate tissue, and then using a stimulus to stimulate a material of the closure device to weld and interdigitate the approximated tissue, and then to release tissue weld strengthening agents.

FIGS. 3A-3C includes images that show different suture strands.

FIG. 4 includes an image that shows a suture in a knot, which provides evidence of use as a functional suture.

FIG. 5A includes an image that shows a suture with the stimulus responsive material.

FIG. 5B includes an image that shows a suture without the stimulus responsive material.

FIG. 6 includes a graph that shows photothermal response of collagen-GNR fibers exposed to pulsed wave (PW) or continuous wave (CW) near infrared light at varying power densities.

FIG. 7 includes a graph showing representative curves of collagen-GNR fibers extended by 4%, 16%, and until breaking at 0.25 mm/min.

FIG. 8 includes a graph that shows ultimate tensile strength of collagen-GNR fibers compared to commercially available PGA sutures (n=5).

FIG. 9 includes a graph that shows representative stress-strain curves of PGA sutures and collagen-GNR fibers extended at a rate of 1 mm/min until failure.

FIG. 10 includes a graph that shows burst point pressure of intestinal samples. Incised cylindrical tissue sections were welded as described previously.

FIG. 11 includes a graph that shows the ultimate tensile strength of various materials of monofilament closure materials.

FIG. 12 includes a graph that shows the ultimate tensile strength of various materials of double filament closure materials.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology relates to materials that can be included in devices for wound closure and healing. The materials can be included in closure devices, such as sutures, staples, or the like. The materials can be included in closure devices so as to achieve stimuli responsive tissue-integrating closure devices (e.g., sutures/staples) that can provide for rapid tissue closure in surgical wounds (e.g., incisions) and injury wounds. The materials can be responsive to a stimulus, such as photothermal excitation (e.g., laser/light excitation), to enhance the ability of a closure device to close a wound for improved healing. The use of these materials in a closure device combines the benefits of both tissue approximation (e.g., suturing) healing and external stimulus triggered tissue welding (e.g. photothermal tissue welding), and direct tissue integration. The materials can include a biocompatible material that is configured as a closure device supplemented with stimulus responsive nanoparticles. The biocompatible material can be a structural material that provides structure to the closure device and that includes the stimulus responsive nanoparticles. In one example, the stimulus responsive nanoparticles can be responsive to photothermal excitation in order to provide an additional benefit of enhancing wound closure via photothermal tissue welding; however, nanoparticles that are responsive to other stimuli can be used. The stimulus responsive nanoparticles can convert light into heat, such as by plasmon resonance or other phenomena. This allows the closure device to be used conventionally, and to be treated with a stimulus (e.g., photothermal excitation) to enhance wound closure and healing. However, the nanoparticles may have different compositions that are simulated by different stimuli as described herein.

The closure device can include tissue integrating material technology for accelerated wound repair. Tissue integrating materials may also be referred to as stimuli responsive tissue integrating suture materials or STISMs, and both terms are used in this document. The STISMs can be used in various closure devices (e.g., sutures/staples) in order to allow for the closure device to respond to an external or internal stimulus after tissue approximation to seal the tissue in response to the stimulus (e.g. light or magnetic stimulus). These closure devices can provide improved performance in facilitating repair of the wounded tissue due to their dual properties of mechanical strength and stimulus triggered tissue integration. By integrating with the surrounding tissues, these closure devices (e.g., biodegradable) generate a homogenous weld/seal across the injury. Alternatively, when the device (e.g., biostable staple) does not integrate with the tissue, the tissue on each tissue portion can integrate together. The resultant healed wound is more stable and is less prone to wound dehiscence and leakage (or other problems) than obtained from other wound healing techniques.

The closure device having the stimuli responsive nanoparticles can provide a number of improvements. The nanoparticles can be selected to be responsive to a particular stimulus so that the closure device can integrate with the tissue upon exposure to a defined stimulus or cause tissue integration. The nanoparticles that are added to the material of the closure device do not impair the mechanical properties of the closure device. After tissue approximation, the closure device can be stimulated to induce integration of the material of the device with the tissues. The closure device may also be stimulated to induce integration of the tissues that are pulled together with the closure device in order to close an open wound, with or without the closure device integrating with the tissue. The closure device can include nanoparticles that generate heat when subjected to the stimulus in order to initiate tissue welding and in some instances cause closure device integration via heat generation, which leads to protein interdigitation or chemical reaction with the tissue. The closure device can provide dual benefits that include accurate tissue approximation by the structure of the closure device followed by stimuli responsive tissue welding and integration, which can increase mechanical stability of the wound closure that can lead to rapid healing.

The ability to enhance tissue closure can allow for new methods of stitching wounds with a suture that does not leave material behind, such as once a suture is removed. Often sutures are installed with knots. However, the sutures having the improved material with the stimulus response nanoparticles may be installed without knots by cinching the tissue of the wound closed and then using the stimulus to facilitate interdigitation and wound closure. As such, knots may be omitted, and thereby removal of the suture may not result in portions of the suture (e.g., knots) being left in the tissue. Knots and other suture materials left behind after conventional wound closure can cause a body to produce an immunological reaction to the foreign body.

The material can include the nanoparticles at various sizes, concentrations, amounts, distributions, or arrangements in the structural material. The modulation of the nanoparticles in size, amount, or type can be used to control the response to the stimulus. In one example, when the nanoparticle generates heat in response to the stimulus, the control of the nanoparticles can be used to control the heat generation from the stimulus. As such, the control of the nanoparticles can provide accurate control of heat generation or stimuli responsiveness. Also, the methods of use can include modulating the power or intensity or time of application of the stimulus to modulate the heat generation. The modulation of the stimulus can be conducted during the surgical procedure, where the temperature of the wound and/or closure device can be monitored with a temperature monitoring device, and the application of the stimulus can be modulated in order to modulate the temperature. Modulation of the stimulus may be conducted along with modulation of the inclusion of the nanoparticles in the structural material.

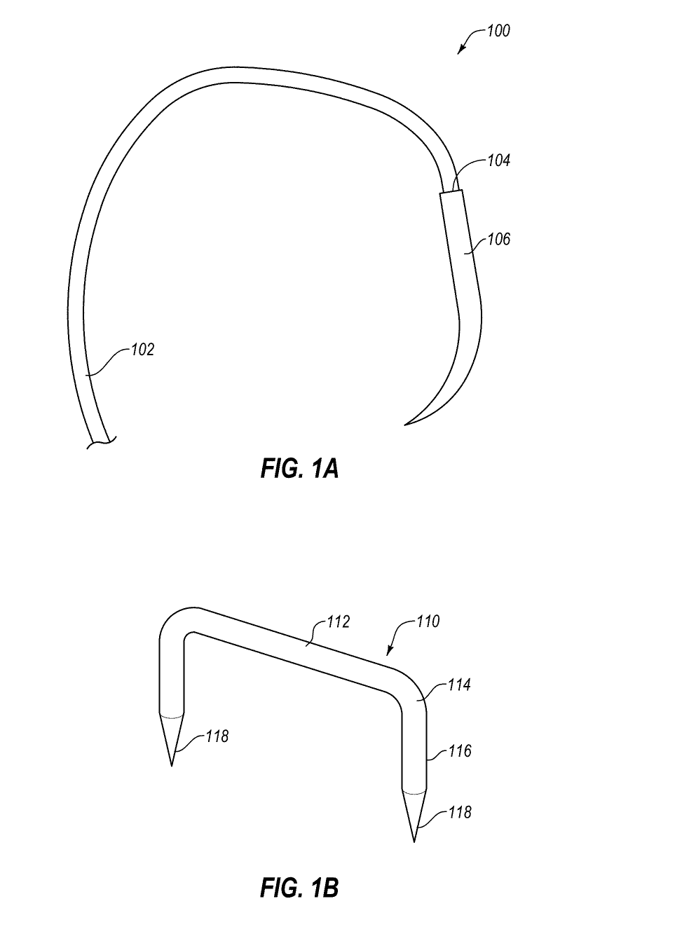

The closure devices described herein can be made by various processes known in the art. In fact, common manufacturing can be used to prepare a suture (FIG. 1A) or a staple (FIG. 1B). FIG. 1A shows a surgical suture 100 having a suture cord 102 that is coupled to a needle 106, through a suture end 104. Here, the suture cord 102 is configured with the stimulus responsive material. FIG. 1B shows a surgical staple 110 having a cross-member 112 with bends 114 at each end, which bends 114 have down-members 116 with tine ends 118. Here, the surgical staple 110 can include the stimulus responsive material.

The closure devices can be made in various ways depending on whether a suture, staple, or other is used. As such, manufacturing protocols can be adapted to include forming the body of the closure device to include the stimulus responsive material. In an example for a suture, a solution of collagen with dispersed stimulus responsive materials (e.g., gold nanorods (GNRs)) is extruded into a fiber formation buffer, incubated in an additional buffer, and hung to dry and extend under the tension of their own weight. The collagen solution concentration, GNR weight percent, extrusion rate, and inner diameter of extrusion tubing can be all varied to produce a wide range of diameter fibers.

Additionally, the use of the stimulus can allow for accurate and precise control of the tissue integration process to avoid any unnecessary inflammation, fluid influx, and neutrophil extravasation that could compromise the weld strength. The protocol can include modulating the stimulus to reduce the action if any inflammation, fluid influx, and neutrophil extravasation is observed.

In one embodiment, the nanoparticles may be responsive to two or more stimuli, such that one or more stimuli can be applied to obtain the response from the nanoparticle. In one aspect, two or more different nanoparticles may be included in the structural material that are responsive to two or more different stimuli. As such, a first nanoparticle may be responsive to a first stimuli, and a second nanoparticle may be responsive to a different second stimuli. This can allow for using one or two different stimuli to induce the wound closure and tissue healing. The different stimuli can be used at the same time, different times, in sequence, or in patterns to promote enhanced wound closure.

In one embodiment, the structural material can be biodegradable and/or bioabsorbable. This can allow for the closure device to be degraded and possibly excreted from the body. The nanoparticles may also be biodegradable and/or bioabsorbable. This can allow for the closure device to be easily removed by the body after prolonged exposure to bodily fluids, cells, or other substances.

In one embodiment, the device, such as structural material or coating, may also include a biologically active agent, such as a drug. In one example, the device can include an antimicrobial (e.g., antibiotic) that can inhibit infections in the wound. Such active agents can enhance the healing. As such, the closure device can be configured to allow drug elution from the closure device before, during, and after tissue integration from the stimulus, which can provide for faster healing of the wound tissue.

In one embodiment, the closure device (e.g., sutures/staples) composed of a biocompatible material (e.g., polymer) has nanoparticles (e.g., inorganic) that are sensitive to a stimulus. The biocompatible material of the closure device can be a structural material that provides the structure of the closure device. The nanoparticles can provide the stimulus sensitivity to the closure device. The nanoparticles can be on the surface of the closure device, embedded in the structural material, retained in the structural material with the ability to translocate therein, fixed in the structural material in discrete locations, or otherwise included with the structural material. The nanoparticles can be encapsulated within a network of the structural material or can be crosslinked with the structural material. The nanoparticles may covalently bond with the structural material or be otherwise associated therewith.

In one embodiment, the nanoparticles (e.g., gold) can convert incident light (wavelength: 650-1350 nm) energy to heat by plasmon resonance, or collective oscillation of free electrons in the nanoparticle. The generated heat, upon reaching a critical temperature (e.g., 50-70 degrees C.), can result in protein structural changes in the tissue. Such structural changes can cause the tissue of the wound to adhere so as to close the opening of the wound. The protocol can include annealing the tissue having the closure device in order to induce the adherence of the tissues. The protocol can use annealing (e.g., heating and then slow cooling) so that the tissue proteins interdigitate, and together with the suture result in rapid sealing of the wound opening in the tissue. In addition, the closure device (e.g., suture/staple) becomes integrated with the tissue to generate a robust uniform weld/seal of the wound opening.

While light stimulus has been described, the nanoparticles may have different compositions that are sensitive to different triggers and/or stimuli. Some examples of stimuli that can induce the nanoparticles to respond and facilitate wound closure can be provided by electrical, thermal, chemical, mechanical, magnetic, acoustic, pressure, shear, or other stimulus that is applied to the tissue or body (e.g., external stimuli). However, the triggers and/or stimuli may be provided by the body (e.g., source inside the body) from biological molecules, administered substances, catalytic actions, enzymatic actions, or chemical reactions in order to initiate wound closure (e.g., weld or seal). As such, the nanoparticles in the closure device can be stimulated to weld/seal apposing wound edges. In each of these cases, the closure device can be specially designed to respond to a specific external stimulus (e.g., optical, magnetic, electrical, or thermal source) or internal stimulus (e.g., human body's enzymatic, catalytic, or chemical reactions). Upon response to the stimulus, the closure device can integrate with the opposing tissue edges of a wound, and thereby close the wound and weld/seal the tissue.

The materials having the nanoparticles can be used in various closure devices that are configured to close a wound in a tissue. In one example, the closure device is a suture, and may be used in surgical procedures. Suturing alone (e.g., in many surgeries of the gastrointestinal tract) to approximate tissues (e.g., two portions of a tissue on opposite sides of a wound) does not result in immediate tissue sealing; wound leakage or dehiscence occurs in many cases. In colorectal surgery, for example, leakage is a feared complication and results in life-threatening consequences. Rapid tissue closure has the potential to mitigate these challenges. Laser tissue welding for rapid tissue closure has been researched using a wide variety of techniques and procedures. Briefly, laser tissue welding has been performed using laser light at mid or far infrared wavelengths to excite the natural chromophores within tissue. Endogenous laser tissue welding, as it is referred to, while successfully closing ocular and other tissue in a number of cadaver studies is only applicable to highly homogenous, thin, and transparent tissue. However, laser tissue welding may cause damage to the tissue at or around the wound. Now with the inclusion of stimulus responsive nanoparticles in the closure device, the present technology provides for suturing to approximate the tissues and then using the stimulus to weld the tissue together. Herein, instead of the laser tissue welding, a stimulus (e.g., laser light) can be used to excite the nanoparticles that in turn cause heat generation that facilitates the tissue welding. Thus, the use of approximation and stimulus responsive tissue welding that can control heat generation and temperature reduces the likelihood of peripheral thermal damage to the surgical area of tissue and surrounding tissue. Accordingly, the present technology is an improvement over a combination of suturing and laser tissue welding.

In one embodiment, the closure device can include materials of gold nanorod (GNR)-elastin-like polypeptide (ELP), which can be included in the structural material. Alternatively, the elastin-like polypeptide may be used as the structural material. Also, the material can include a GNR-collagen nanocomposite configured as a closure device. The GNR-collagen can convert near infrared (NIR) laser to heat (photothermal activity). The photothermal GNR-collagen is a light absorbable nanocomposite that can be shaped into the closure device or included in the structural material of the closure device (e.g., sutures/staples) and used as described herein for tissue repair.

Nanocomposite materials for photothermal tissue repair were generated and investigated using a small intestine anastomosis surgery model. Collagen-gold nanorod hydrogel films were extruded as fibers/sutures. The nanocomposite hydrogel fibers converted near infrared light to heat, and were determined to be comparable in strength to commercially-available multifilament braided sutures. Currently available sutures only act as threads woven through the tissue and to approximate apposing edges, and they do not seal the tissue, and thus lower the mechanical stiffness of the tissue after approximation. It has now been found that the tissue integrating sutures having the nanoparticles can be used with stimulus triggers to improve wound closure by tissue welding and integration. As one example, the gold nanorod-based absorbable sutures are more efficient than organic chromophores/dyes in converting incident laser light into heat, thereby welding with the tissue by inducing protein interdigitation to produce a strong tissue weld. Upon welding, these sutures also integrate with the tissue to produce a robust tissue weld. Thus, the closure devices having the stimulus responsive nanoparticles can be used to enhance healing and result in robust tissue welds that seal tissues.

The closure devices can have various configurations. For example, the stimulus responsive nanoparticles can be included in: sutures, whether extruded, monofilament, multifilament, braided, and of any suitable material whether biostable or biodegradable; absorbable synthetic sutures; antimicrobial (e.g., triclosan) sutures; drug-eluting sutures; staples (e.g., with or without the properties of sutures described herein); wound adhesives; or any other wound closure device.

The closure devices (e.g., sutures or staples) with the stimulus responsive nanoparticles can provide sufficient support to properly seal the torn/injured soft tissues after surgery or accident. These stimulus responsive sutures and staples can be used to bring two tissue ends together before being simulated to promote improved tissue sealing and healing. The stimulus responsive closure devices can inhibit tissue dehiscence and leakage of contents into the surroundings from a wound or tissue opening.

In the first embodiment, photoresponsive absorbable sutures are provided that respond to incident near-far infrared laser light (e.g., 650-1350 nm). These photoresponsive sutures can include, but are not limited to, a biocompatible cross-linked polymer embedded or crosslinked with inorganic nanoparticles, chemical dyes, drugs, or other components. These nanoparticles/chemical dyes can convert externally provided near infrared laser light to heat by excitation-emission, plasmon resonance, or collective oscillation of free electrons. The generated heat upon reaching a critical temperature (e.g., 50-70 degrees C.) results in structural changes in tissue proteins in contact with the photoresponsive absorbable sutures that are being directly irradiated, and the proteins of the apposing edges of a wound or other tissue opening are interdigitated and fuse together. After the stimulus is no longer provided, there is a lowering in temperature that results in rapid tissue welding/sealing. In this process, the suture/staple matrix polymer also interdigitates and bonds with the tissue proteins to generate a gapless integration.

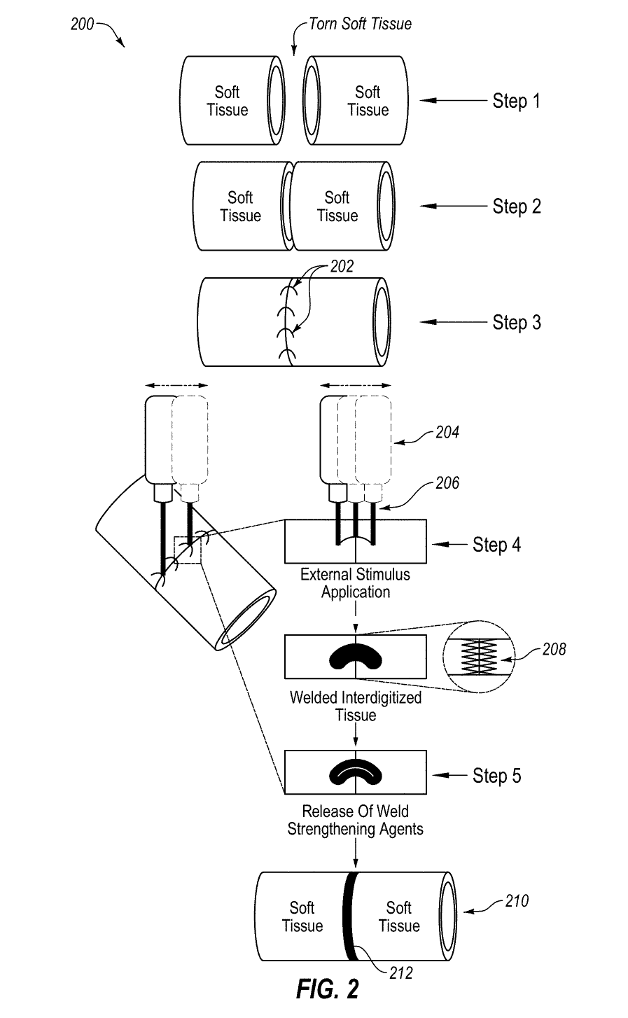

A schematic representation of application of the STISM for tissue welding via tissue integration is shown in FIG. 2. FIG. 2 shows a series of steps 200 to prepare welded tissue. Step 1 having soft tissues (e.g., tissue portions) that are separated by a gap (e.g., torn soft tissue). Step 2 shows that the soft tissues are firstly brought into proximity with each other. Step 3 shows that the soft tissues are approximated using STISMs 202 so that the tissues become connected to each other through the ECM rich region (e.g., Sub-mucosa in colon) of the soft tissues. After proper tissue approximation, Step 4 shows the stimulus is provided by a laser 204 that emits stimulating laser light 206 to the STISMs 202 to initiate generation of heat. The heat causes tissue welding via protein interdigitation 208. Step 5 shows that the weld strengthening agents are released after welding and act to strengthen the weld and promote tissue healing 210 with the soft tissues having the tissue weld 212.

The closure device can have various configurations. Some examples can include monofilament and braided sutures and bodies formed into staples made of biocompatible natural/semi-natural/synthetic polymeric materials (i.e., structural materials) including: nylon, rayon, polyethylene, F127 (poloxamer 407), chitosan, collagen, laminin, fibronectin, polyacrylamide, aminoglycoside hydrogels, fibrin, poly-lactic acid, poly-glycolic acid, polyglyconate (Maxon), polydioxanone (PDS), poly-lactic-co-glycolic acid, silk, poly-glycolic-caprolactone, cotton, gelatin, polypropylene (prolene), and many others. Titanium metal, non-absorbable plastic (nylon, polypropylene, polyester and polysulfone) or absorbable staples (made using homopolymers and copolymers of lactide, glycolide and p-dioxanone) may also be coated with such polymeric coatings having the stimulus responsive nanoparticles. The structural materials can be coated, embedded or crosslinked or otherwise associated with various stimuli responsive materials (e.g., nanoparticles and/or organic/inorganic dyes) or other materials (e.g., drugs). These closure devices allow for initial tissue approximation followed by tissue welding and suture integration into the tissue when triggered by the appropriate external/internal stimuli. A blood vessel closure device, such as a cauterizer, may also be configured as described herein. The STISMs may also be combined with medical grade adhesives, sealants, or hemostatic components.

Various external (source outside the body) and/or internal (source inside the body) stimuli that can be applied to stimulus responsive closure devices can include: optical (e.g., light), electrical, thermal, chemical, mechanical, magnetic, acoustic, pressure, shear, biological, or enzymatic. The stimulus application can be sufficient to initiate the closure device to weld/seal apposing wound edges of a tissue. These external/internal stimuli can induce crosslinking of the proteins/polypeptides/fats that are in contact with or close to the closure device by either: (1) increasing temperature leading to a phase change in proteins/polypeptides for interdigitation; (2) initiating a chemical reaction; or (3) physically or chemically interacting with the tissue nearby in any other way. The end result achieved after exposure of the tissue integrating sutures to the stimulus is a robust and a rapid tissue welding. Further, the suture/staple also integrates with the tissue to generate a uniform and robust weld.

Examples of materials that can be responsive to an optical (e.g., light) stimulus can include: gold nanorods, gold nanoparticles, gold nanospheres, indocyanin green, neodymium-doped nanoparticles (Nd:NPs), carbon nanotubes (CNTs), organic nanoparticles (O:NPs), gold nanostars (GNSs), or near-infrared absorbing dyes (absorbance of the dye between 650-1350 nm). Many materials have a range of wavelengths to which they are responsive, and may be tuned to a specific wavelength.

In one embodiment, laser light energy is converted to heat (e.g., photothermal conversion). Photoresponsive tissue integrating sutures or staples (or other closure device) are generated by adding and/or reinforcing and/or doping and/or coating the closure device material (e.g., biocompatible natural and/or semi-natural and/or synthetic polymers) with light (e.g., wavelength-650-1350 nm) absorbing elements. The light stimulus response materials can advantageously absorb in the optical window of 650-1350 nm light wavelength and convert the laser energy into heat. The heat produced causes a physico-chemical change in the tissue (e.g., in the immediate vicinity of the closure device) leading to interdigitiation (e.g., protein/polypeptide/fat fusion) of two ends of the tissue that results in tissue welding. The suture/staple also integrates with the tissue to generate uniform weld.

In one embodiment, magnetic energy is converted to heat (e.g., magnetothermal). A closure device (e.g., monofilament or multi-thread braided tissue integrating sutures or staples) can include particles that convert magnetic energy to heat by adding and/or reinforcing and/or doping and/or coating the closure device material (e.g., biocompatible natural and/or semi-natural and/or synthetic polymers) with a biocompatible particle. The particle can be organic dyes, inorganic dyes, or organic nanoparticles or inorganic nanoparticles, or ferromagnetic particles or anti-ferromagnetic particles (e.g., 1-100 nm longest dimension) that absorb the incident magnetic field to produce heat and/or initiate a chemical reaction which allows for tissue welding and sealing by protein interdigitation or chemical reaction between a suture-tissue and tissue-tissue junction. The closure device can provide for tissue integrating sutures that allow for tissue approximation followed by tissue welding. The closure device also integrates with the tissue to generate uniform weld.

In one embodiment, electrical energy is converted to heat (e.g., electrothermal). A closure device (e.g., monofilament or multi-thread braided tissue integrating sutures or staples) can include particles with resistive elements that can convert electrical energy into heat or and/or initiate a chemical reaction which allows for tissue welding and sealing by protein interdigitation or chemical reaction between a suture-tissue and tissue-tissue junction. The closure device can provide for tissue integrating sutures that allow for tissue approximation followed by tissue welding. The closure device also integrates with the tissue to generate uniform weld. The particles having the resistive elements can be included in the closure device by adding and/or reinforcing and/or doping and/or coating the closure device material (e.g., biocompatible natural and/or semi-natural and/or synthetic polymers) with a biocompatible particle having the resistive element. The resistive element can be any organic dyes, inorganic dyes, or organic nanoparticles or inorganic nanoparticles, or ferromagnetic particles or anti-ferromagnetic particles (e.g., 1-100 nm longest dimension) that absorb the electrical energy and convert the electrical energy as described herein.

In one embodiment, an internal stimulus is used to induce the tissue welding. The closure device (e.g., monofilament/multi-thread braided tissue integrating sutures or staples) includes the closure device material (e.g., biocompatible natural and/or semi-natural and/or synthetic polymers) with a biocompatible particle having the ability to facilitate tissue welding upon exposure to an internal stimulus. The internal stimulus can be blood, blood components, native moisture/water, or amines and/or hydroxyls and/or carboxyl groups in the proteins, glycans, or other features of the native tissue). In one example, the closure device can be coated, conjugated, or treated with substances or particles to expose terminal free aldehyde or epoxy groups which can interact with the amines and/or hydroxyls and/or carboxyl groups of the native proteins in the tissue allowing for tissue welding. This can facilitate a chemical reaction which allows for tissue welding and sealing by protein interdigitation or chemical reaction between a suture-tissue and tissue-tissue junction. The closure device can provide for tissue integrating sutures that allow for tissue approximation followed by tissue welding. The closure device also integrates with the tissue to generate a uniform weld. In another example, the closure device can be configured to expose terminal free fibrinogen or thrombin groups which can interact with the blood component in the closure device incision site of the tissue that can cause or provide tissue welding.

In one embodiment, the particles can be protein-based nanoparticle composites that can be responsive to a stimulus as described herein. The protein-based nanoparticle composites may be self-responsive to the stimulus or include a material described herein as being responsive to the stimulus. Such protein-based nanoparticle composites can be included in the structural material of the closure device.

In one embodiment, a tissue-integrating closure device described herein that is responsive to a stimulus can elicit low inflammation and decrease the chance of wound dehiscence and rupture. The stimuli-responsive closure device can be used for rapid tissue closure creating a fluid-tight seal immediately upon conclusion of the stimuli exposure treatment. For example, photothermal sutures that are laser-responsive sutures can be used for rapid tissue closure and creating a fluid-tight seal immediately upon conclusion of the laser treatment. The particles that convert the stimulus to heat can be used to kill bacteria due to heat, thereby decreasing the likelihood of infection.

In one embodiment, the closure device can include drugs that can elute into the body, where the drugs can elute from the structural material or coating or other part as is common with drug eluting devices, or the particles can be susceptible to degradation by exposure to the stimulus in order to induce the drug elution. Accordingly, drug-elution from the closure device can be a response to the external or internal stimuli exposure.

In one embodiment, the closure device can include specific moieties that can allow for delayed activation and triggering of tissue welding to allow for proper tissue approximation by the surgeon prior to stimuli response. Also, protocols to generate heat can be performed where the materials respond and produce heat when photothermally activated.

In one embodiment, the structural material can provide structural integrity to the closure device. The particles having the stimuli sensitive materials can be included into the closure device in an amount and/or distribution that does not lower its mechanical properties. The amount and/or distribution of the particles or stimuli sensitive material can be modulated in order to achieve the stimulus response as well as retain the structural integrity of the closure device.

In one embodiment, the closure device can include a combination of stimulus responsive materials (e.g., in particle form or molecular form) distributed in the structural material so that two or more stimuli can be used for enhanced tissue welding. In one aspect, a combination of internal and external stimuli can be used to initiate the tissue welding for accurate tuning of a sequence of events leading to tissue welding. For example, a fibrinogen coated suture that is reinforced with photothermal elements can use internal stimuli (e.g., blood) as well as external stimuli (e.g., light) for welding. First, the fibrinogen can react with the blood and then light can be applied as desired to induce the photothermal effect. As such, sequential stimuli can be applied in a desired order to enhance tissue welding. This allows for only one stimuli or more than one stimuli to be used.

In one embodiment, the structural material of the closure device can be biodegradable or bioabsorbable so that the closure device can be easily removed after exposure to body fluids or cells. This can provide for absorbable tissue integrating closure devices for wound repair, which can allow for accurate tissue approximation followed by rapid tissue welding and sealing in response to an external and/or internal stimulus. The closure device can provide for a tighter weld or seal to a torn, cut, or ruptured tissue than conventional sutures/staples/tissue adhesives and sealants due to the dual benefits of (1) mechanical stabilization (tissue approximation) and (2) tissue integration (tissue welding). Additionally, the mechanical or stimulus responsive properties can be tuned easily by adjusting the weight percent of polymer of the structural material and adjusting the weight percent of the stimuli responsive organic/inorganic additives (e.g., particles, molecules, or substances).

The closure devices described herein have a number of advantages over common closure devices or common tissue laser welding. In one aspect, the closure device includes gold nanorods that have much higher quantum yield than that of dye based systems, where the gold nanorods can convert a much higher percentage of the incident laser light into heat. This can reduce laser exposure times and provide higher efficiencies compared to laser welding. The laser density can be less than laser welding, where the inventive closure devices received laser at 2.5 W/cm², which is the lowest recorded power density used for this application. This low power density can increase efficiency, reduce side effects, and lower costs. The lower laser density also achieved 68% and 64% recovery in tissue tensile strength and tissue burst pressure after the welding. The closure device can include a solid matrix that can be molded into different shapes apart from suture or staples, such as a patch and fibers or others, which allows for the closure device to be provided in a number of configurations to provide the benefits described herein.

In one embodiment, common closure devices (e.g., sutures, screws, staples, patch, or other) can be coated with a coating having the stimulus responsive materials to provide the benefits described herein. As such, prior to use, the stock closure device can be immersed into a solution having the stimulus responsive materials and optionally dried before being used to approximate tissues and receiving the stimulus. Sealants, such as tissue adhesives (e.g., cyanoacrylates), can be doped with the stimulus responsive materials to provide the benefits described herein.

While the STISM is described to be configured into nanoparticles, the particulate size may vary to be micron sized (e.g., 1-100 microns or 0.01-1 microns), nano sized (e.g., 1-100 nm or 0.01-1 nm), or smaller. Also, particles may include the STISM as well as other materials, and thereby the STISM particles may be less than 100% STISM (e.g., from 1-100%, 1-99%, 1-90%, 1-80%, 1-70%, 1-60%, 1-50%, 1-40%, 1-30%, 1-25%, 1-20%, 1-10%, or 1-5%). The amount may vary depending on the material. Also, the STISM may be in small particulate form that is distributed through the structural material, such as a homogenous distribution or a gradient that has higher concentration on the outside of the structural material with lower concentration internally, or vice versa.

In one embodiment, the technology includes a three-pronged approach to achieve strong stimuli initiated tissue welding and sealing using the suture/staple materials described herein. This three-pronged approach includes: (1) first tissue approximation by suturing; (2) second heat generation by stimulus response for tissue integration; and (3) third bond strengthening by additional agents. The materials that can incorporate all three of these aspects are referred to as stimuli-responsive tissue-integrating suture/staple material. However, the third approach (3) may be omitted in some instances.

In an example, the stimuli-responsive tissue-integrating suture/staple material (STISM) is first used to approximate the apposing ends of the torn/wounded tissue. The suture/staple is run through the layer of the tissue that is rich in collagen, fibronectin, and other ECM connective tissue components (e.g., sub-mucosa in colon) to approximate the apposing ends of the torn tissue. Next, an external energy stimulus comprising of any of the following: electrical, thermal, optical, magnetic, or acoustic energy, specific to the absorbing element in the STISM is applied to the site of the suture/staple and sutured/stapled tissue. The heat is then transferred to the tissue, and interdigitation of proteins occurs to seal the tissue and STISM together. During recovery, weld strengthening components act on the treated tissue to improve healing. The weld strengthening components can be active agents that promote wound healing.

The STISM contains stimulus-responsive elements (nanomaterials, organic or inorganic dyes, microparticles, or particulates) that respond to an external stimulus. This stimulus is applied via an external source (e.g., source outside the body) and/or a catheter/endoscope near the site of the wound/injury. The energy stimulus is absorbed by the stimulus responsive element in the STISM and converted into heat. The heat generated in the STISM is transferred to the approximated tissue, and causes a physico-chemical phase change to begin breaking of hydrogen linkages in the tissue proteins/fat in order to cause them to interdigitize. Once the external stimulus is removed, the phase change is reversed, resulting in strengthening of the interdigitation of the adjacent apposing tissue ends and the STISM material and a uniform continuous welded tissue.

In a specific embodiment of this technology, a collagen-gold nanorod (collagen-GNR) STISM upon exposure to near-infrared light causes gold nanorod mediated production of heat. Gold nanorods absorb and convert the incident near-infrared (NIR) laser light into heat (NIR is weakly absorbed by the tissues). The heat induces a phase change in the collagen protein of the STISM and the tissue (gel-to-sol) leading them to interdigitize. After removal of the near-infrared light source, the interdigitation is strengthened as the tissue and STISM cool resulting in a uniform tissue weld, having integration between the apposing ends of the tissue with the STISM. A successful completion of this process results in complete tissue interdigitation, fusion, and integration of the STISM to the tissue.

The third component of the suture/staple material (STISM) includes one or more special components that can strengthen the weld by either initiating a secondary chemical reaction/chemical interaction within the welded tissue or inhibiting the biological processes that work to weaken the weld. These sutures/staples are functionalized with, or coated in, components such as antibiotics to inhibit bacterial growth or release specific MMP inhibitors (e.g., polyvinylpyrrolidone (PVP), doxycycline, Cefoxitin, broad spectrum antibiotics, etc.) to reduce anastomotic leakage or anti-inflammatory drugs (e.g., aceclofenac, acemetacin, aspirin, celecoxib, dexibuprofen, dexketoprofen, diclofenac, etodolac, etoricoxib, fenoprofen, flurbiprofen, ibuprofen, indometacin, ketoprofen, mefenamic acid, meloxicam, nabumetone, naproxen, sulindac, tenoxicam, and tiaprofenic acid) that can reduce the biological processes working to weaken the weld. The STISM could also be loaded with drugs by various techniques such as physical entrapment, surface modification, coating, or crosslinking to create chemo-attractive gradient for stem cell chemotaxis to initiate/accelerate the repair of the wound.

In Aspect 1, the STISM is used in a traditional wound closure application as either a suture or staple to approximate tissue/wound edges. According to the current state of the art, the STISM may be a braided multifilament or monofilament suture, an absorbable or non-absorbable suture, an antibacterial or untreated suture, and a conventional or knotless spiral anchor suture, as well as other wound closure devices or compositions. The STISM may be a suture composed of a biological polymer or protein such as chitosan, fibrin, elastin, collagen, or silk, or a synthetic polymer such as polypropylene, polyglycolic acid, vicryl, or nylon, or even a metal, such as stainless steel, with a stimulus-responsive element located therein or thereon or otherwise associated therewith. The STISM may be a metal staple made of stainless steel or titanium coated in a stimulus-responsive composite, or may be an absorbable or non-absorbable staple composited of a synthetic and/or absorbable polymer or protein, such as polyglycolic acid, polylactic acid, or polydioxanone, with a stimulus-responsive element associated therewith.

Using the STISM, tissue approximation may be performed using any technique used in conventional stapling/suturing/knot tying, such as simple interrupted suturing, simple continuous suturing, over and over suturing, horizontal and vertical mattress suturing, or lock-stich suturing.

In Aspect 2, in addition to the conventional staple/suture component of the STISM, each STISM includes a stimulus-responsive component. Specific examples of stimuli and stimulus-responsive elements (SRE) are given herein. These SREs may be embedded in a matrix (e.g., polymer matrix) or encapsulated or otherwise distributed (e.g., homogeneous or in a concentration gradient) within the suture/staple component by various means such as physical entrapment, crosslinking, core-shell extrusion, chemical conjugation, dissolution, or may coat the suture/staple component.

The general mechanism of STISMs in response to an external stimulus is to generate heat and provide heat to tissue as follows. The heat generated from an external stimulus causes a physico-chemical change in the tissue (e.g., in the immediate vicinity) and interdigitiation (e.g., protein/polypeptide/fat fusion) of two ends of the tissue either with themselves or with the closure material (e.g., suture/staple). The proposed mechanism (e.g., known as tissue welding or stimulus-assisted tissue repair) is three-fold. First, at local temperatures exceeding 40° C. collagen fibrils in the tissue becomes less structured and rigid and more fluid and disorganized. Second, at local temperatures exceeding 50° C. intermolecular bonds in the tissue proteins are broken and frayed, resulting in interdigitation with the proteins/polymer of apposing tissue and the STISM material. A similar response occurs in the STISM polymer organization, though the corresponding temperatures of these two steps may be different than for the tissue. Third, as the stimulus is removed and the local temperature decreases, these interdigitated polymers/protein bond and are strengthened, resulting in a robust tissue-tissue and/or tissue-STISM bond.

Aspect 3 uses weld strengthening elements added to the STISMs to produce a stronger tissue weld. Various weld strengthening elements can be added to the sutures/staples or other closure device to provide further added support to the weld site after the welding. These weld strengthening elements could include bacteriostatic antibiotics, quaternized polymers or bactericidal antibiotics or silver nanoparticles that inhibit bacterial growth at the wound site, broad-spectrum small molecule MMP inhibitors, MMP inhibiting polymers, MMP-9 targeted selective inhibitors, MMP-targeted monoclonal antibodies to prevent anastomoses leakage, and anti-inflammatory drugs to prevent large scale inflammatory response at the site of the suture. Bacterium enterococcus faecalis has recently been implicated to activate host MMP-9 activity and cause anastomotic leakage. STISMs with specific MMP-9 inhibitors could suppress MMP-9 host activity and prevent anastomotic leakage after photothermal tissue welding. For example, CAS 1177749-58-4 is a specific MMP-9 inhibitor, which could be blended with polymer solution prior to suture extrusion to generate MMP-9 inhibiting STISMs. Alternately, to prevent very high levels of macrophage and other immune cell infiltration whenever necessary, we propose using oligopeptides that bind the cryptic sites in denatured collagen and other ECM proteins (Patent-WO2011049810A2). These peptides will actively block the cryptic sites that are involved in initiating macrophage and other immune cell infiltration to the site of the weld that will result in reduction in the number of macrophages infiltrating the wound site.

### EXAMPLE 1

### Preparing Stimulus Responsive Closure Device

An example of an experimental protocol showed that the tissue integrating sutures (e.g. photothermal sutures—that integrate in the tissue upon exposure to light) improved wound closure and healing. Type 1 rat tail collagen extracted from rat tail is dissolved in 0.5 M acetic acid at concentrations varying from 5 to 25 mg/ml. A gold nanorod dispersion of varying nanorod concentration in nano-filtered water is added to the collagen solution in various volumes, resulting in a collagen solution with 1 wt % to 10 wt % gold nanorods. Using a syringe pump, the collagen-gold nanorod solution is extruded into a saline suture extrusion buffer. The rate of extrusion and size of extrusion tubing can be adjusted to alter the diameter of the extruded fibers. Collagen-gold nanorod sutures are extruded into an aqueous saline fiber extrusion buffer composed of 118 mM PBS, 20 wt % 8k polyethylene glycol, and pH adjusted to 7.5 at 37° C. and incubated in this buffer for 60 minutes. Following the suture extrusion buffer, the sutures are transferred to an isopropanol buffer at room temperature and incubated for 8 hours. The sutures are then washed in nano-filtered water for 1 hour. After washing, the sutures are removed from the water bath and hung from the center around a plastic rod 5 cm in diameter, resulting in fiber length of approximately 5-20 cm on each side of the hanging rod. The sutures are left hanging to stretch and dry for at least 8 hours to generate photoresponsive tissue integrating sutures. These nanocomposite tissue integrating sutures can be used in a similar fashion to conventional surgical sutures and then stimulated to provide the enhanced wound closure and healing. These nanocomposite tissue integrating sutures have been used to close ex vivo porcine intestine by means of a simple, interrupted suture technique using a surgeon's tie. The sutured site is exposed to laser light corresponding to the maximum absorbance of the photoresponsive element, resulting in its absorbance, generation of heat, elevation of temperature, protein interdigitation, and rapid tissue welding of the injured site.

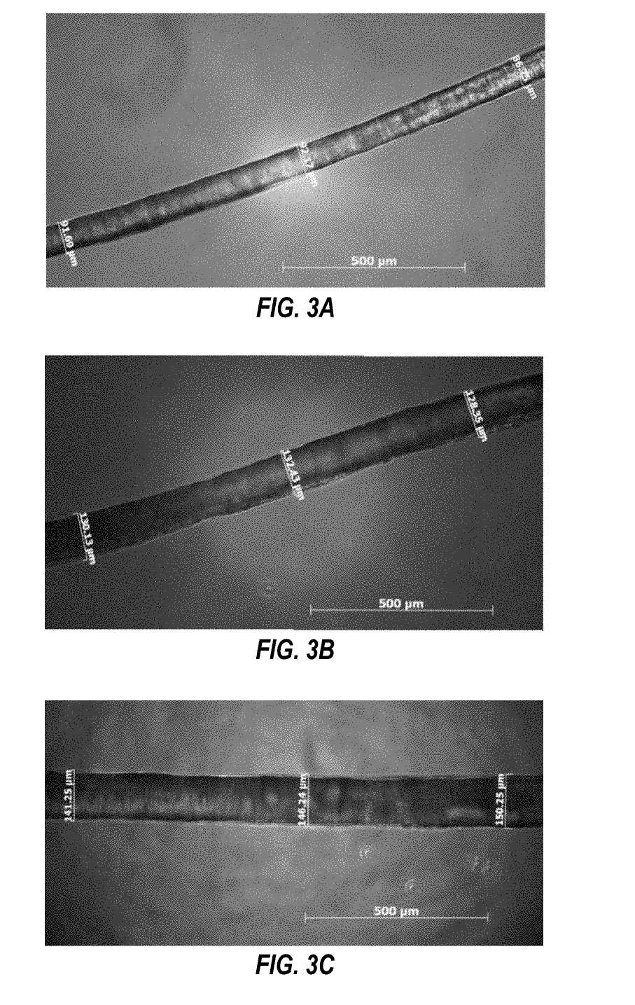

FIGS. 3A-3C include representative microscopic images of Collagen-GNR fibers processed at (FIG. 3A) low, (FIG. 3B) med, and (FIG. 3C) high extrusion rates (scale bar=500 nm). These are images of collagen-GNR fibers that were processed at extrusion rates of (FIG. 3A) 0.2 mL/min, (FIG. 3B) 0.4 mL/min, and (FIG. 3C) 0.6 mL/min. Diameters were taken at three different points of five images of distinct locations along the length of the fibers to determine an average diameter so that commercial sutures of comparable size can be used for determining effectiveness and to attempt to form collagen-GNR fibers of uniform diameter.

Accordingly collagen-GNR fiber diameter is a function of extrusion rate, extrusion tubing diameter, and collagen concentration. Collagen-GNR fibers were extruded under a number of different conditions to vary diameter and determine the optimal parameters for strength and handling. Flow rates varied at 0.2, 0.4, or 0.6 mL/min; collagen concentration varied from 15, 20, to 25 mg/mL; and extrusion tubing varied in diameter from 0.2, 0.35, and 0.5 mm. The average fiber diameter varies from approximately 150 to 275 μm, corresponding to sutures of size 6-0 to 8-0 USP.

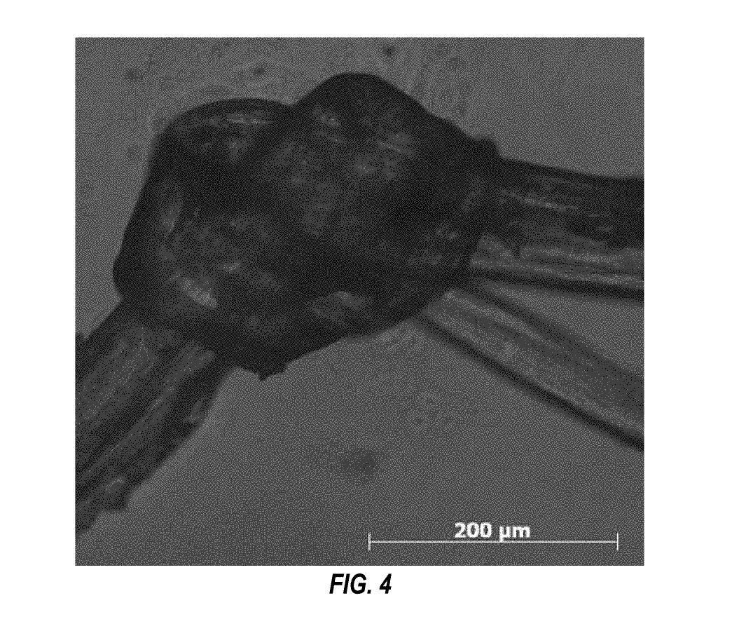

FIG. 4 includes a microscopic image of a collagen-GNR fiber in a suture knot (scale bar=200 nm). Fibers were typically used to make suture knots using a surgeons' tie and three additional single ties.

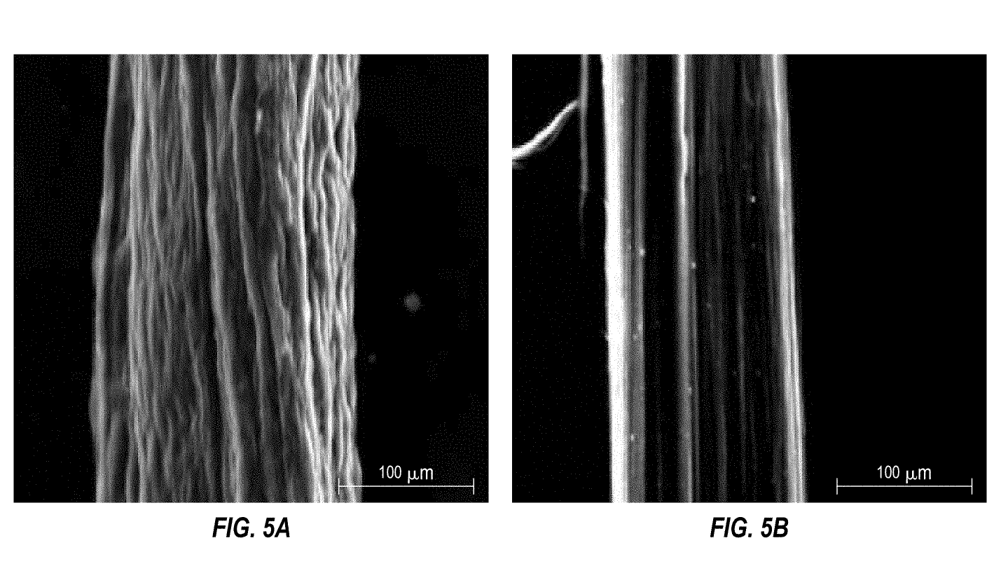

FIGS. 5A-5B include scanning electron micrographs of the surface of collagen fibers (FIG. 5A) with and (FIG. 5B) without GNRs (scale bar=100 μm). As shown, there is a structural or morphological difference when GNRS are included.

### EXAMPLE 2

### Laser/Light Energy is Converted to Heat (Photothermal)

Photoresponsive tissue integrating sutures/staples (STISMs) are generated by adding/reinforcing/doping/coating the biocompatible natural/semi-natural/synthetic polymers with a light (e.g., wavelength-650-1350 nm) absorbing elements including, but not limited to, gold nanorods (GNRs) (e.g., maximum absorbance ranging from 700 to 1300 nm); gold nanoparticles (e.g., wide range of absorbance); gold nanospheres (e.g., maximum absorbance at approximately 520 nm); neodymium-doped nanoparticles (e.g., wide range of maximum absorbances); carbon nanotubes (e.g., maximum absorbance ranging from 600 to 1400 nm); organic nanoparticles (O:NPs) such as polyaniline (e.g., maximum absorbance at 775 nm) or polypyrrole nanoparticles (e.g., maximum absorbance at approximately 540 nm); and gold nanostars (e.g., maximum absorbance ranges from 700 to 900 nm); or near-infrared absorbing dyes such as indocyanine green (e.g., maximum absorbance at 800 nm), methylene blue (e.g., multiple absorbance peaks with a maximum at approximately 670 nm), india ink (e.g., absorbance in India ink in the range of 400-1100 nm), and rose-bengal dye (e.g., maximum absorbance at 560 nm). All of these above materials absorb in the optical window of 500-1350 nm light wavelength and convert the laser energy into heat. The proposed mechanism of action for photothermal tissue sealing is the same as described above.

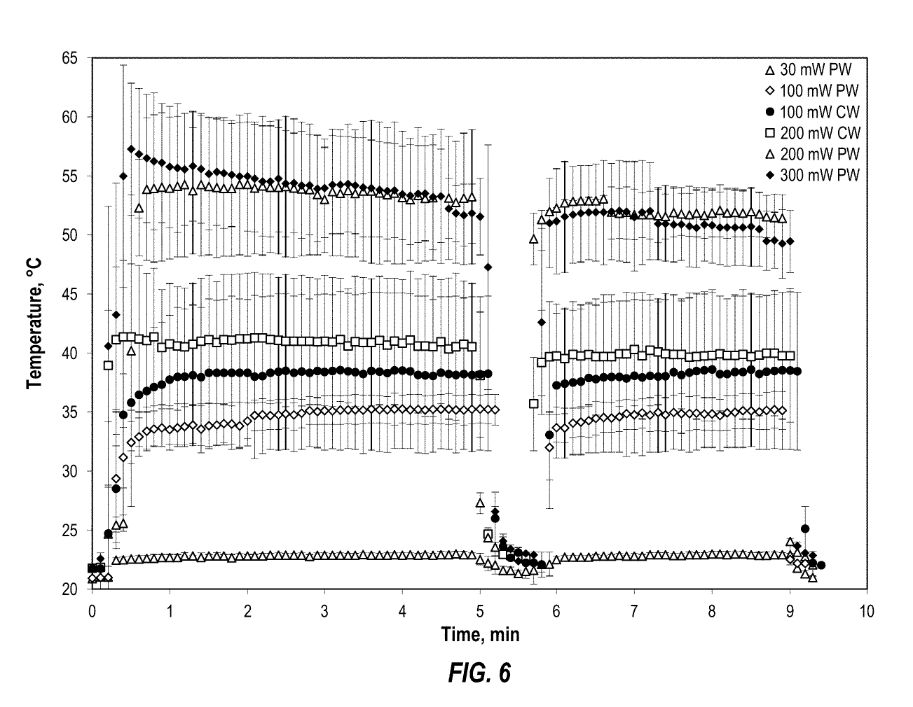

FIG. 6 includes a graph that shows photothermal response of collagen-GNR fibers exposed to pulsed wave (PW) or continuous wave (CW) near infrared light at varying power densities. n=3. Each fiber/suture is placed on a glass slide and irradiated with the laser for 4 continuous minutes; the laser shutter is then closed for 30 seconds. Afterwards, the fiber/suture is again irradiated for 4 minutes, with an additional 30 seconds of no laser exposure. During these approximately 9 minutes, an infrared camera is positioned directly above the experimental area outside of the laser beam path. The camera records infrared heat profiles of the surface of the fiber/suture every 4-5 seconds, and the maximum temperature reached on the fiber/suture surface is recorded in the plot in FIG. 6. As seen from these results, the maximum temperature reached is directly dependent on the power density of the laser irradiation applied. The fibers generate heat from the laser exposure very quickly and approach a maximum temperature within at most one minute of laser exposure. Additionally, the temperature drops rapidly back to room temperature in the absence of the laser, and the maximum temperature reached during the second exposure does not seem to be altered by the first exposure, suggesting no loss in the effective heat generation. These attributes all contribute to limiting thermal damage by minimizing the area that is heated and minimizing the time of heating. Laser powers at 200 mW and higher begin to approach the maximum temperature necessary for robust tissue welding to occur. Of the laser power densities tested, 300 mW pulsed wave reached the maximum temperature at approximately 55 C.

### EXAMPLE 3

### Magnetic Energy is Converted to heat (Magnetothermal)

The monofilament/multi-thread braided tissue integrating sutures or staples (STISMs) are composed of a biocompatible natural/semi-synthetic/synthetic polymer added/doped/reinforced with magnetically-responsive elements such as organic/inorganic magnetothermal dyes, iron oxide-pNIPAAM nanoparticles, Strontium Doped Lanthanum Manganite Nanoparticles, amphiphilic block copolymers coated Mno_{0.6}Zno_{0.4}Fe₂O₄ nanoparticles, Magnetothermally responsive star-block copolymeric micelles (star-block copolymer poly(ε-caprolactone)-block-poly(2-(2-methoxyethoxy)ethyl methacrylate-co-oligo(ethylene glycol)methacrylate) and Mn, Zn doped ferrite magnetic nanoparticles (MZF-MNPs)) (e.g., 1-100 nm longest dimension), Poly(hydroxyethyl methacrylate) (PHEMA) and poly(N-i sopropylacrylamide-co-acrylamide) P(NIPAAm-co-AAm) coated FePt, Fe₃O₄ and CoFe₂O₄ nanoparticles and other ferromagnetic magnetothermal substances that absorb the incident magnetic field generated via RF coil to produce heat to initiate protein interdigitation in the apposing tissue ends leading to tissue sealing. Like the previous sutures/staples, this embodiment of tissue integrating sutures/staples allows for tissue approximation followed by tissue welding. The suture/staple also integrates with the tissue to generate a uniform weld. Specific examples of additive materials that can absorb incident magnetic energy to generate heat are described herein. The proposed mechanism of action for magnetothermal tissue sealing is the same as described above.

### EXAMPLE 4

### Electrical Energy is Converted to Heat (Electrothermal)

The monofilament/multi-thread braided tissue integrating sutures or staples (STISMs) are composed of a biocompatible natural/semi-synthetic/synthetic polymer added/doped/reinforced with electrically resistive elements such as organic/inorganic dyes or nanoparticles (e.g., 1-100 nm longest dimension) such as tin-doped indium oxide nanoparticles, carbon nanotubes, graphene nanosheets, silver nanoparticles, gold nanoparticles, polyaniline nanoparticles, barium titanate nanoparticles, bismuth telluride nanoparticles, other resistive substances) that can convert the electrical energy into heat/initiate a chemical reaction which allows for tissue welding/sealing by protein interdigitation/chemical reaction between STISM-tissue/tissue-tissue junction. Like the previous sutures/staples, this embodiment of tissue integrating sutures/staples allows for tissue approximation followed by tissue welding. The suture/staple also integrates with the tissue to generate a uniform weld. The proposed mechanism of action for electrothermal tissue sealing is the same as described above.

### EXAMPLE 5

### Acoustic Energy is Converted to Heat (Acoustothermal)

The monofilament/multi-thread braided tissue integrating sutures or staples (STISMs) are composed of a biocompatible natural/semi-synthetic/synthetic polymer added/doped/reinforced with polymer or lipid shelled microbubbles, which could generate heat under focused ultrasound energy transmitted via a transducer head. The microbubbles could generate heat under different acoustic power sources (e.g., 0.6-20 W). The microbubbles solution could be combined with the polymeric solution and extruded into sutures or staples or coated thereon.

Like the previous sutures/staples, this embodiment of tissue integrating sutures/staples allows for tissue approximation followed by tissue welding. The suture/staple also integrates with the tissue to generate a uniform weld. The proposed mechanism of action for acoustothermal tissue sealing is the same as described above.

### EXAMPLE 6

### Thermal Energy is Transmitted Through the Suture/Staple Material

Commercially existing stainless steel sutures could be connected to an external heating element to transfer heat to the site of the suture. The heat dissipation from the suture could trigger collagen interdigitation, tissue integration, and welding of the injury site. In addition, polymeric materials (biocompatible natural/semi-synthetic/synthetic polymers) could be braided with stainless steel sutures to conduct heat energy to weld the tissue. The suture/staple could also merge with the tissues/cause tissue integration upon heating. The proposed mechanism of action for thermal tissue sealing is the same as described above.

### EXAMPLE 7

### Internal Stimuli to Crosslink the Extracellular Matrices of Torn Tissues

Although heat based welding is used as an example to seal adjacent pieces of tissues, there could be other ways to connect the connective matrix of two pieces of torn tissues. Connective tissues of adjacent torn soft tissue segments could be connected by a polymeric gel that can selectively crosslink with the amine and caroboxylic groups exposed in the proteins. In addition, protein/peptide based hydrogels could form a bridge between the sub-mucosa (or other ECM rich tissue layer) of two torn tissue segments such that macrophages, fibroblasts, stem cells and other immune-cell migration, chemotaxis, and infiltration could be achieved. Collagen-GNR based STISMs are an embodiment of this approach. Collagen, being the most abundant protein in the body, could effectively serve as the bridge between the ECM rich layers of the two sub-mucosa of the torn tissue sections (or other ECM rich tissue layer). Other proteins such as laminin, fibronectin, hyaluronan, etc., could be used in the same way.

A safe, biocompatible, polymer based crosslinker that mimics the ECM of native tissue could be used to connect the two ECM rich layers of the torn tissue segments leading to cell infiltration and further wound healing.

### EXAMPLE 8

### Preparing Stimulus Responsive Closure Device

Rat tail tendon collagen extraction is performed. Tendons were removed from humanely collected rat tails and type 1 atelocollagen was extracted similar to previous procedures using pepsin/acid solubilization.

Gold nanorods (GNRs) synthesized according to the seed-mediated growth method, forming nanorods stabilized by a CTAB bilayer. GNRs tuned to ˜800 nm maximum absorbance, determined by a Biotek Synergy 2 plate reader. Centrifugation, decanting, and water redispersion performed as described previously.

Collagen-gold nanorod nanocomposite fiber extrusion is performed. Lyophilized collagen was dissolved in 0.5 M acetic acid at a concentration of 10, 15, or 20 mg/mL. GNRs were added to these viscous collagen solutions, resulting in 5, 10, or 15 wt % GNR dispersions. Following the addition of GNRs, the collagen solutions were raised to a pH of ˜5.0 and mixed at 4° C. for 24 hours to allow GNR-crosslinking with the collagen.

Similar to other procedures, collagen was extruded through thin tubing using a syringe pump into a polyethylene glycol (PEG, 8K) buffer solution. In short, a 3 mL syringe (Terumo) connected to 8-12 inches of tubing was loaded onto a syringe pump (NE 300, New Era Pump Systems, Inc). The tubing varied at 1.5, 3.0, or 4.5 mm inner diameter. The pump was set to a flow rate of 0.2, 0.4, or 0.6 mL/min, and the tubing flowed into a small container holding fiber extrusion buffer (FEB) at 37° C. FEB was 110 mM phosphate buffer and 20 wt % PEG10. Following 60 minutes in FFB, the fibers were incubated in isopropanol for an additional eight hours. The fibers were then transferred to a distilled water bath and finally hung and air dried at room temperature under tension for at least 12 hours.

The fibers were then cross-linked by dehydration at elevated temperatures. The fibers were incubated for 24 hours at 40° C. Collagen is degraded enzymatically at a very high rate in the body, and chemical crosslinking can give collagen materials stability throughout the implantation period. 1-ethyl-3-(3-dimethylaminopropyl)carbodiimide (EDC) was used to intrafibrillarly crosslink collagen without toxic side products as follows. Extracted collagen at 10 g/mL with 6 mmol EDC for 18 hours at room temperature. The amount of carboxylic acid groups was approximated by assuming that each alpha chain is 100,000 Da with 120 COOH groups per alpha chain. The EDC to COOH group ratio was 1:1 or 1:2.

In one embodiment, the monofilament/multi-thread braided tissue-integrating sutures or staples are composed of biocompatible natural/semi-synthetic/synthetic polymers that have been modified to interact with the tissue using its internal stimulus (blood or blood components or native moisture/water or amines/hydroxyls/carboxyl groups in the proteins/glycans of the native tissue).

In one embodiment, the suture/staple can be coated/conjugated/treated to expose terminal free aldehyde/epoxy groups which can interact with the amines/hydroxyls of the native proteins in the tissue allowing tighter integration of the weld after thermal interdigitation of the surrounding connective tissue.

In one embodiment, the suture/staple can be coated/conjugated/treated to expose terminal free fibrinogen/thrombin groups which can interact with the blood component in the suture/stapling incision site of the tissue allowing for tissue sealing after thermal interdigitation of the surrounding connective tissue.

In one embodiment, collagen-binding peptides such as bone morphogenic protein 2 and hyaluronan-binding peptides can be added to the STISMs allowing tighter integration of the weld after thermal interdigitation of the surrounding connective tissue.

In one embodiment, a protein-based nanoparticle composite can be applicable for sutures of any kind.

In one embodiment, tissue-integrating sutures/staples which elicit low inflammation and decreasing chance of wound dehiscence and rupture can be used.

In one embodiment, drug-elution from the suture can be adapted to occur as a response to external/internal stimuli exposure. For example, a stimulus, such as a laser, can cause the body of the suture (or other closure device) to become degraded and allow the drug to elute therefrom. The laser may form pores in the body of the closure device to allow the drugs to elute therefrom.

In one embodiment, inclusion of specific moieties can be included in the suture device that can allow for delayed activation and triggering of stimuli responsive elements or delayed release/activation of weld strengthening agents to allow for proper tissue approximation by the surgeon prior to stimuli response.

In one embodiment, incorporation of stimuli-sensitive materials into the suture does not lower its mechanical properties. That is, the mechanical properties are sufficient for use in medical procedures to promote wound healing as described herein.

In one embodiment, a combination of internal and external triggers/stimuli together or individually could be used to initiate the tissue welding after tissue approximation followed by weld strengthening. For, example, fibrinogen coated suture -reinforced with photothermal elements that can use internal as well as external stimuli for welding.

In one embodiment, smart sutures/staples that can be triggered by more than one external or internal stimuluses. For example, STISMs coated/reinforced with gold nanorods coupled with iron oxide nanoparticles could generate heat after being exposed to laser light and/or magnetic RF source simultaneously or individually.

In one embodiment, the sutures or staples described herein are bioabsorbable and can be easily removed by the body after prolonged exposure to bodily fluids/cells or the like.

In one embodiment, commercially existing suture materials such as PGA, PLGA, poliglecaprone, polycaprolactone-PGA, polycaprolactone-PLGA sutures could be reinforced with stimuli responsive materials and weld strengthening agents to convert them into STISMs for tissue approximation, welding and further weld strengthening.

In one embodiment, the weld strengthening agents can be resistant to the external stimuli that are being provided to the STISMs to initiate tissue welding and would not lose any of their biological activity after exposure to heat or any stimuli specific to the STISMs.

In one embodiment, bioabsorbable stimuli responsive tissue integrating suture/staple materials (STISMs) are provided for wound repair that can allow for accurate tissue approximation followed by rapid tissue welding and sealing in response to an external/internal stimulus. Further, the weld strengthening agents loaded into these STISMs would allow for faster healing and recovery of the wound. This category of materials improves weld/seal integrity to a torn/ruptured tissue compared to conventional sutures/staples/tissue adhesives and sealants due to the dual benefits of mechanical stabilization (tissue approximation) and tissue integration (tissue welding).

In one embodiment, the STISM and application of the stimulus can be utilized with: triclosan-coated sutures; staples; fibrin glue; sealants and adhesives; albumin solder or other solders for laser tissue welding.

A protocol was performed to determine the strength of a suture closure device having laser-responsive (e.g., stimulus being a laser) material. This allows for the suture to be used to approximate the tissues, and then a laser welding process that directs the laser to the tissue-integrating sutures so that the laser-responsive material causes the material to facilitate welding. An incision is made in a slab of intestinal tissue. The incision is closed with a GNR-collagen suture and irradiated with an NIR laser (e.g., see FIG. 2). The incision is sealed following laser exposure. The intestine is then clamped at both edges and pulled until failure while measuring the force and distance. This experiment is used to determine the ultimate tensile strength of the incised tissue following closure and will be compared to the strength of intact tissue, incised tissue with no treatment, and incised tissue sutured conventionally.

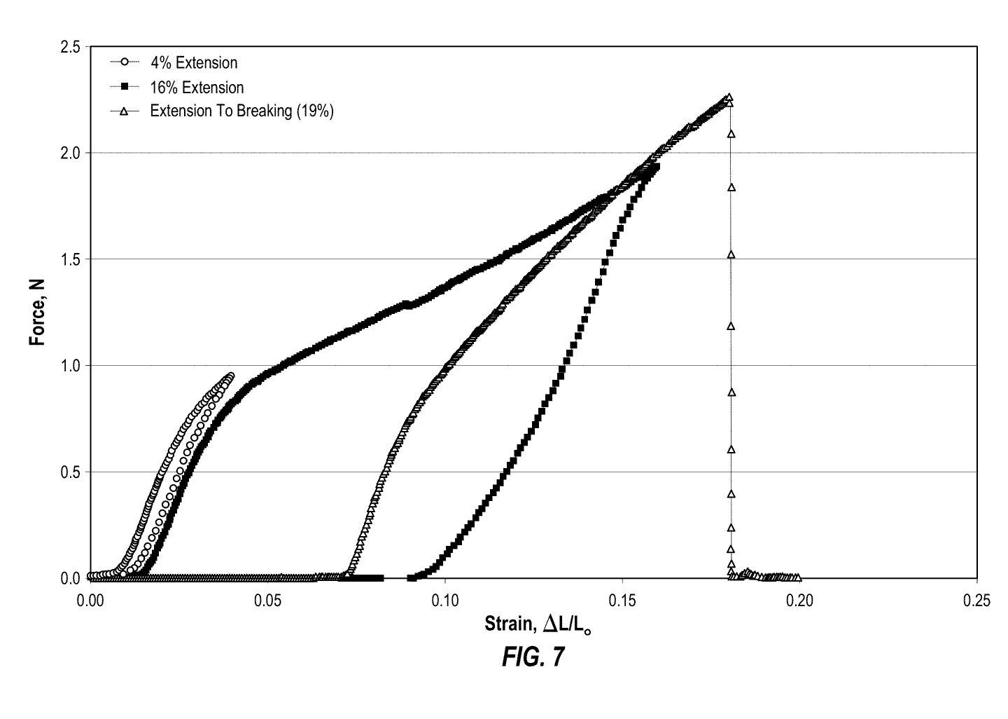

FIG. 7 includes a graph showing representative curves of collagen-GNR fibers extended by 4%, 16%, and until breaking at 0.25 mm/min. Fibers and sutures were loaded into clamps and extended to a target distance while measuring force under tension until finally failing at approximately 19% extension. These data show that the fibers are not brittle. Curves are representative of n=3 independent experiments.

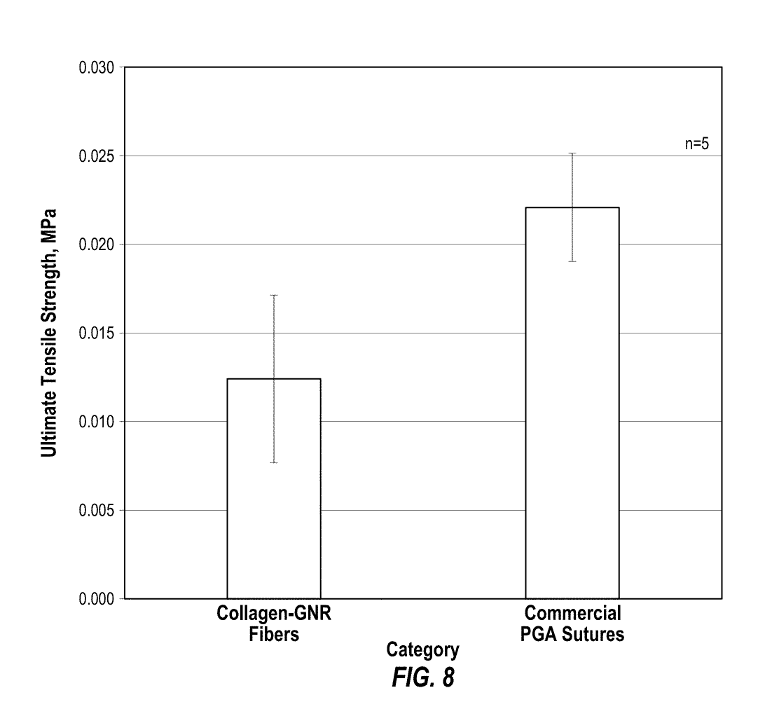

FIG. 8 includes a graph that shows ultimate tensile strength of collagen-GNR fibers compared to commercially available PGA sutures (n=5). Ultimate tensile strength of collagen-GNR fibers compared to commercially available PGA sutures of similar diameter. Fibers or sutures of similar length are clamped at each edge and extended at a rate of 1 mm/min until failure, and the ultimate tensile strength was recorded in each case. From these results, we see that monofilament collagen-GNR fibers have roughly half the ultimate tensile strength of braided commercially-available PGA sutures.

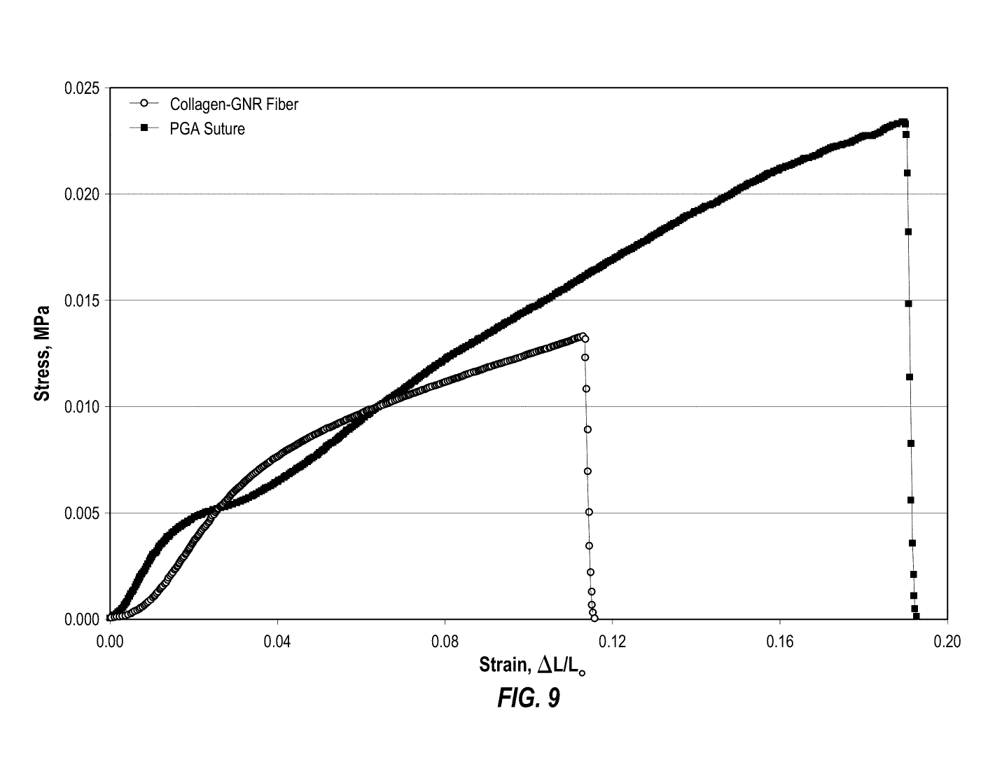

FIG. 9 includes a graph that shows representative stress-strain curves of PGA sutures and collagen-GNR fibers extended at a rate of 1 mm/min until failure. Again, monofilament collagen-GNR fibers have approximately half the strength of braided commercially-available PGA sutures of similar diameter. Curves representative of n=3 independent experiments.

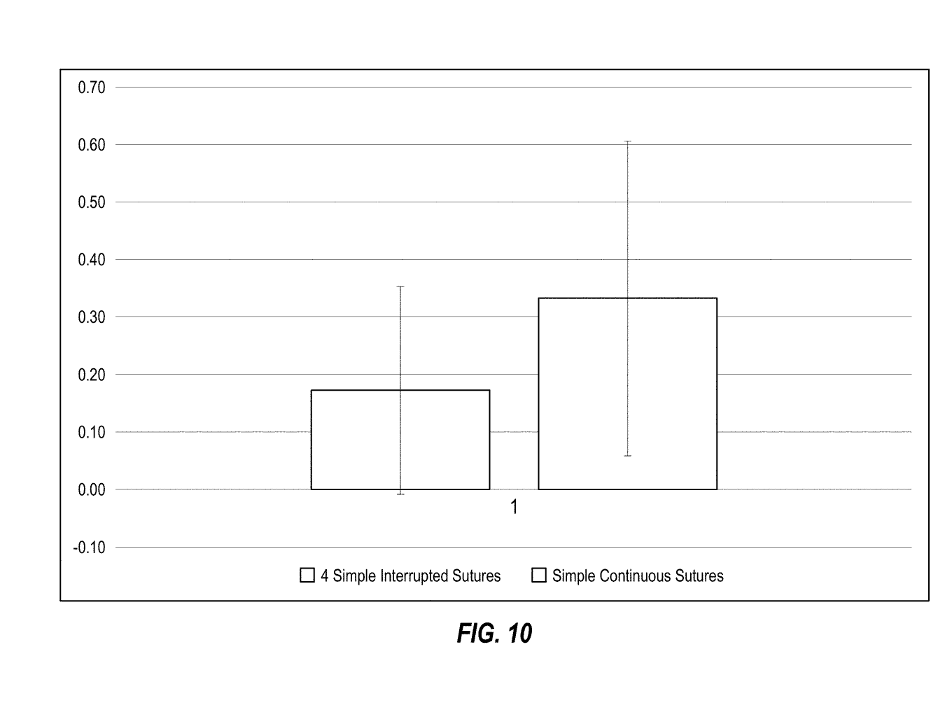

FIG. 10 includes a graph that shows burst point pressure of intestinal samples. Incised cylindrical tissue sections were welded as described previously. Following welding, the tissue was clamped closed at both ends and infused with a saline solution while measuring the fluid pressure. The maximum pressure reached before leakage or bursting was recorded. Intestine conventionally sutured using two different common and relevant suture techniques both show very small maximum burst pressures of the closure (n≧5).

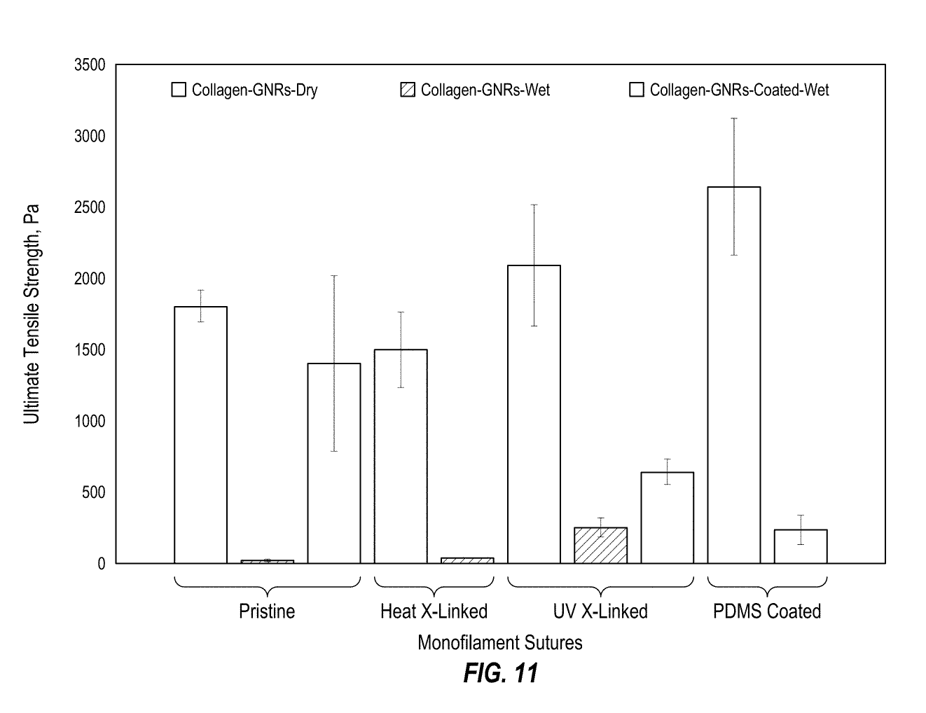

FIG. 11 includes a graph that shows the ultimate tensile strength for different monofilament sutures, such as collagen-GNRs when dry, collagen-GNRs when wet, and coated collagen GNRs that are wet, when exposed to different stimuli or not exposed to stimuli. Pristine is when not exposed to stimuli. Heat X-linked is when exposed to a stimulus that causes heat. UV X-linked is when exposed to UV light. PDMS coated is when coated with PDMS.

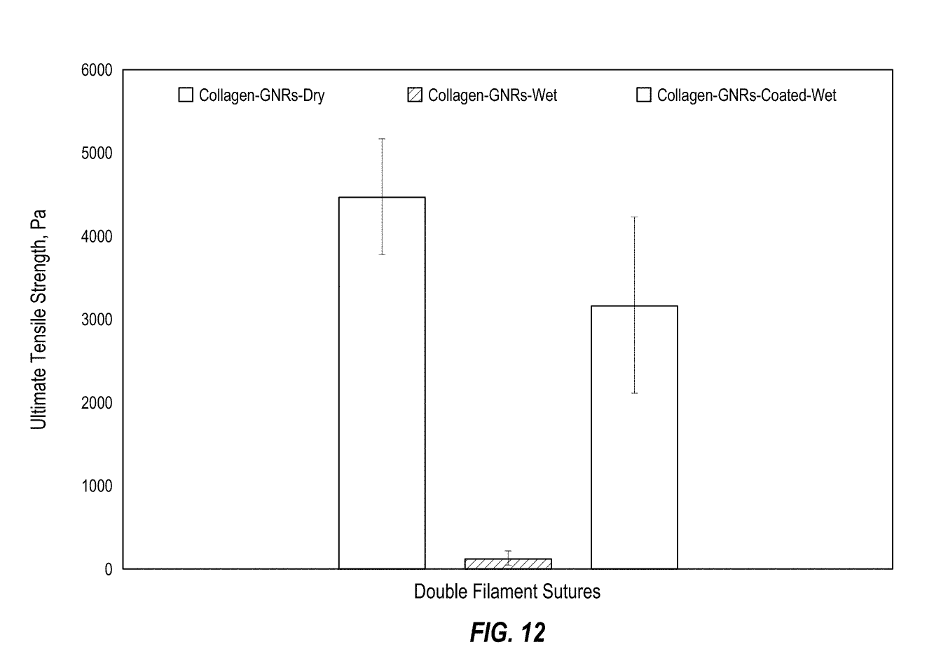

FIG. 12 includes a graph that shows the ultimate tensile strength for different double filament sutures, such as collagen-GNRs when dry, collagen-GNRs when wet, and coated collagen GNRs that are wet.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions, or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims. All references recited herein are incorporated herein by specific reference in their entirety.

1. A tissue closure device comprising:
a body having a structural material; and
a stimulus responsive material on or in the structural material.

2. The device of claim 1, wherein the structural material is biodegradable and/or bioabsorbable.

3. The device of claim 1, wherein the structural material includes nylon, rayon, polyethylene, pluronic F127, chitosan, collagen, laminin, fibronectin, polyacrylamide, aminoglycoside hydrogels, fibrin, poly-lactic acid, poly-glycolic acid, poly-lactic-co-glycolic acid, polyglyconate, polydioxanone, silk, poly-glycolic-caprolactone, cotton, gelatin, polypropylene, titanium, metal, polysulfone, copolymers thereof, or combinations thereof.

4. The device of claim 1, wherein the stimulus responsive material is a photoresponsive material that is stimulated by light.

5. The device of claim 4, wherein the stimulus responsive material is selected from gold nanorods, gold nanoparticles, gold nanospheres, gold nanostars, indocyanin green, neodymium-doped nanoparticles, carbon nanotubes, organic nanoparticles, or near-infrared absorbing dyes having absorbance between 650-1350 nm, and combinations thereof.

6. The device of claim 1, wherein the stimulus responsive material is a magnetically responsive material that is stimulated by magnetic energy.

7. The device of claim 6, wherein the stimulus responsive material is selected from organic dyes, inorganic dyes, organic nanoparticles, inorganic nanoparticles, ferromagnetic particles, or anti-ferromagnetic particles that absorb an incident magnetic field.

8. The device of claim 1, wherein the stimulus responsive material is an electrically responsive material that is stimulated by electricity.

9. The device of claim 8, wherein the stimulus responsive material is selected from electrically resistive organic dyes, inorganic dyes, organic nanoparticles, inorganic nanoparticles, ferromagnetic particles, or anti-ferromagnetic particles that convert electricity to heat.

10. The device of claim 1, wherein the stimulus responsive material is a chemically responsive material that is stimulated by blood, blood components, water, amines, hydroxyls, or carboxyl groups.

11. The device of claim 10, wherein the stimulus responsive material is selected from substances having exposed aldehydes or epoxy groups that react with amines, hydroxyls, or carboxyl groups of proteins.

12. The device of claim 1, wherein the stimulus responsive material is in a particle form.

13. The device of claim 1, wherein the structural material is configured as a suture, staple, screw, patch, adhesive, or sealant.

14. The device of claim 1, further comprising a biologically active agent on or in the structural material.

15. A method of promoting wound healing, the method comprising:
providing a tissue closure device having a body with a structural material and a stimulus responsive material on or in the structural material;
approximating tissue portions of a wound with the tissue closure device; and
stimulating the stimulus responsive material with at least one stimulus so as to cause the tissue portions of the wound to adhere to each other and/or to the tissue closure device.

16. The method of claim 15, wherein the at least one stimulus is selected from optical, electrical, thermal, chemical, mechanical, magnetic, acoustic, pressure, shear, biological or enzymatic.

17. The method of claim 16, comprising stimulating the stimulus responsive material to generate heat that causes tissue components of the tissue portions to interdigitate.

18. The method of claim 15, comprising eluting a biologically active agent from the tissue closure device into the wound.

19. A method of making a tissue closure device, the method comprising:
obtaining a structural material;
obtaining a stimulus responsive material; and
combining the structural material and the stimulus responsive material to form the tissue closure device having the structural material and stimulus responsive material.

20. The method of claim 19, comprising combining a biologically active agent with the structural material.
